(19) United States
(12) Patent Application Publication
(10) Pub. No.: US 2002/0106406 A1
(43) Pub. Date: Aug. 8, 2002

(54) Crystallizable/non-crystallizable polymer composites

(76) Inventors: Anthony J. McHugh, Urbana, IL (US); Jessica R. DesNoyer, Urbana, IL (US)

Correspondence Address: BRINKS HOFER GILSON & LIONE, P.O. BOX 10395, CHICAGO, IL, 60610, US

(21) Appl. No.: 09/733,640

(22) Filed: Dec. 8, 2000

(51) Int. Cl. A61K 9/22

(52) U.S. Cl. 424/468

(57) ABSTRACT

A mixture containing a bioactive agent and one or more biodegradable polymers for use as a biodegradable implant is described. Different ratios of semi-crystalline to amorphous polymers in the mixture provide for different release profiles of the bioactive agent from the implant. This system allows tailoring of the release profile by control of the composition of the implant.

### FEDERALLY SPONSORED RESEARCH OR DEVELOPMENT

The subject matter of this application was in part funded by the National Science Foundation (Grant no. CTS 97-31530). The government may have certain rights in this invention.

### BACKGROUND

Sustained delivery of bioactive agents, especially peptide- and protein-based drug therapies, have been achieved through the use of biodegradable polymeric implants. Traditionally, this technology has involved surgical implantation of a polymeric monolith containing a suspended bioactive agent. Certain complex shapes of these monoliths have been developed to provide a constant release of the bioactive agent over a period of time. This type of release is described as zero-order as the rate of release is not affected by the concentration of the agent. Zero-order kinetics are desirable for therapies that require the administration of a constant level of a bioactive agent. Polymer microspheres encapsulating a bioactive agent can also be used for controlled release and are generally administered by subcutaneous injection. Although their implantation is easier than that of monoliths, the release mechanism of microspheres is rarely zero-order.

Drug release from polymeric implants have been described in U.S. Pat. Nos. 4,938,763; 5,702,716; and 5,733,950 by Dunn et al. These patents describe a solution of biodegradable polymer and bioactive agent in a biocompatible solvent. The solution solidifies upon injection into the body to form a polymeric implant. Release of the bioactive agent is provided by diffusion of the agent from the polymeric matrix, by degradation of the polymer and subsequent release of the agent into the surrounding environment, or by a combination of these two mechanisms. In some instances, this method yields zero-order release kinetics.

Zero-order release kinetics are not ideal for all therapies. For example, vaccinations generally require rapid releases of a bioactive agent at specific intervals. Other therapies require a combination of rapid releases and slow, gradual administration. Thus, there is a need for biodegradable implants that have a variety of drug release characteristics, or profiles.

### BRIEF SUMMARY

In an embodiment of the invention, there is provided a composition for controlled release of a bioactive agent, comprising a biodegradable crystallizable polymer, a biocompatible solvent, and a bioactive agent. The composition may further comprise at least one component solvent, an emulsifying agent, and a biodegradable amorphous polymer. The solvent preferably has a miscibility with water less than 7 percent by weight. The biocompatible solvent is preferably ethyl benzoate. Preferably, the composition is sterile. The biodegradable crystallizable polymer is preferably a polyester and is more preferably poly(ε-caprolactone). The biodegradable amorhpous polymer, if present, is preferably a polyester and is more preferably poly(D,L-lactide). The composition may be multi-layered.

In another embodiment of the invention, there is provided a composition for controlled release of a bioactive agent, comprising a biodegradable crystallizable polymer and a biodegradable amorphous polymer. The composition may further comprise a biocompatible solvent, a component solvent, an emulsifying agent, and a bioactive agent. The solvent preferably has a miscibility with water less than 7 percent by weight. The biocompatible solvent is preferably ethyl benzoate. Preferably, the composition is sterile. The biodegradable crystallizable polymer is preferably a polyester and is more preferably poly(ε-caprolactone). The biodegradable amorphous polymer is preferably a polyester and is more preferably poly(D,L-lactide).

In another embodiment of the invention, there is provided a method of administering a bioactive agent, comprising inserting into an organism a mixture of biodegradable crystallizable polymer, a biocompatible solvent, and a bioactive agent. The inserting may be by injecting. The method may further comprise combining the biodegradable crystallizable polymer and biocompatible solvent with a biodegradable amorphous polymer.

In another embodiment of the invention, there is provided a method of administering a bioactive agent, comprising inserting into an organism a mixture of biodegradable crystallizable polymer and biodegradable amorphous polymer. The inserting may be by injecting. The method may further comprise combining the biodegradable crystallizable polymer and biodegradable amorphous polymer with a bioactive agent.

In another embodiment of the invention, there is provided a kit, comprising a container and a mixture in the container. The mixture comprises a biodegradable crystallizable polymer and a bioactive agent in a biocompatible solvent. The kit may further comprise a unit dosage of the bioactive agent, a biodegradable amorphous polymer, and a syringe. The container may comprise a septum. Preferably, the mixture is sterile.

### BRIEF DESCRIPTION OF THE DRAWINGS

FIG. 1 is a cut-away view of a multi-layer depot.

FIG. 2 is a view of a kit.

FIGS. 3-4 are graphs of results from differential scanning calorimetry.

FIG. 5 is a graph of results from protein release studies.

FIGS. 6-9 are scanning electron micrographs of depots after quenching for 15 days.

FIG. 10 is a graph of results from water absorption studies.

FIG. 11 is a schematic of an injection apparatus for a multi-layer depot.

### DETAILED DESCRIPTION

The present invention includes a mixture containing a bioactive agent and one or more biodegradable polymers for use as a biodegradable implant. Different ratios of semi-crystalline to amorphous polymers in the mixture provide for different release profiles of the bioactive agent from the implant. This system allows tailoring of the release profile by control of the composition of the implant.

The terms semi-crystalline and crystallizable refer to a polymer which has the ability to crystallize into ordered morphologies. This generally occurs due to packing of portions of the chains into regular structures. Semi-crystalline polymers are characterized by a melting temperature, which is the temperature above which the crystalline domains, or crystallites, in the polymer become disordered. The melting temperature (Tₘ) is conveniently measured by differential scanning calorimetry (DSC) as the temperature at which there is an endothermic transition. Once a polymer has crystallized into an ordered morphology, it will yield a pattern of rings, spots, or arcs when analyzed by X-ray diffraction techniques.

The term amorphous polymer refers to a polymer which is not capable of organizing into ordered morphologies. This is evident by an absence of a Tₘ in DSC analysis.

Both amorphous and semi-crystalline polymers are characterized by a glass transition temperature (T_{g}). The T_{g} is the temperature below which the polymer is a rigid glass and above which the polymer is a more flexible material. For example, as the temperature of a polymer is increased through the glass transition region, the polymer may be transformed from a rubbery material to a gum and eventually into a liquid. The T_{g} is conveniently measured by DSC as the temperature at which there is a change in heat capacity of the polymer.

The polymers for use with the present invention are materials which decompose when placed inside an organism. This can be observed as a decline in the molecular weight of the polymer over time. Polymer molecular weights can be determined by a variety of methods including size exclusion chromatography (SEC), and are generally expressed as weight averages or number averages. A polymer is biodegradable if, when in phosphate buffered saline (PBS) of pH 7.4 and a temperature of 37° C., its weight-average molecular weight is reduced by at least 25% over a period of 6 months as measured by SEC.

Mixtures are prepared by mixing the polymer(s) and the bioactive agent with a solvent. It is preferred that the polymers used in the invention are biodegradable. Biodegradable polymers eliminate the need to remove the implant after the bioactive agent has been released, since the polymer will gradually break down and may be metabolized or excreted from the body. Semi-crystalline polymers which are useful for the present invention include polyesters, such as poly(ε-caprolactone), poly(glycolic acid), poly(L-lactic acid), poly(D-lactic acid), poly(hydroxybutryate); polyanhydrides, such as poly(adipic anhydride); poly(para-dioxanone); poly(β-malic acid); polyethylene glycol; and copolymers thereof. Amorphous polymers which are useful for the present invention include polyesters such as copolymers of caprolactone, glycolic acid, lactic acid, and hydroxybutryate; polyamines; polyurethanes; polyesteramides; polyorthoesters; polyacetals; polyketals; polycarbonates; polyorthocarbonates; polyphosphazenes; poly(malic acid); poly(amino acids); polyvinylpyrrolidone; polyhydroxycellulose; chitin; chitosan; and copolymers and mixtures thereof. For the copolymers listed, the presence or absence of crystallinity may depend on the proportions of comonomers present in the polymer, as well as their distribution throughout the polymer chain.

The solvent preferably is biocompatible, forms a viscous gel with the polymer, and controls water uptake into the implant. The solvent may be a single solvent or a mixture of solvents. The term solvent, unless specifically indicated otherwise, means a single solvent or a mixture of solvents. Preferable solvents will substantially restrict the uptake of water by the implant and may be characterized as immiscible in water, that is having a solubility in water of less than 7 percent by weight (wt %). Preferably, the solvents are 5 wt % or less soluble in water; more preferably 3 wt % or less soluble in water; and even more preferably 1 wt % or less soluble in water. Most preferably the solubility of the solvent in water is equal to or less than 0.5 wt %.

Water miscibility may be determined experimentally as follows: Water (1-5 g) is placed in a tared clear container at a controlled temperature, about 20° C., and weighed, and a candidate solvent is added dropwise. The solution is swirled to observe phase separation. When the saturation point appears to be reached, as determined by observation of phase separation, the solution is allowed to stand overnight and is checked again the following day. If the solution is still saturated, as determined by observation of phase separation, then the percent by weight of solvent added is determined. Otherwise more solvent is added and the process repeated. Solubility or miscibility is determined by dividing the total weight of solvent added by the final weight of the solvent/water mixture. When solvent mixtures are used, for example 20% triacetin and 80% ethyl benzoate, they are pre-mixed prior to addition to the water.

Examples of solvents include the lower alkyl and aralkyl esters of aryl acids such as benzoic acid, the phthalic acids, and salicylic acid; lower alkyl esters of citric acid, such as triethyl citrate and tributyl citrate and the like; and aryl, aralkyl and lower alkyl ketones. Among preferred solvents are those having solubilities within the foregoing range selected from (i) compounds having the structural formulas: R¹—C(═O)—O—R² or R¹—C(═O)—R² in which R¹ is aryl or aralkyl, R² is lower alkyl or aralkyl, and R¹ and R² are optionally the same or different, with the proviso that when each of R¹ and R² are lower alkyl, the total carbon atoms in R¹ and R² combined are 4 or more, and (ii) lower alkyl and aralkyl esters of phthalic acid, isophthalic acid and terephtalic acid, and (iii) lower alkyl and aralkyl esters of citric acid. Lower alkyl means straight or branched chain hydrocarbons having 1-6 carbon atoms, optionally substituted with non-interfering substituents. Aralkyl means (lower alkyl)phenyl, for example benzyl, phenethyl, 1-phenylpropyl, 2-phenylpropyl, and the like wherein the alkyl moiety contains from 1-6 carbon atoms. Aryl means phenyl, optionally substituted by non-interfering substituents. Many of the solvents useful in the invention are available commercially (SIGMA/ALDRICH, Milwaukee, Wis.) or may be prepared by conventional esterification of the respective arylalkanoic acids using acid halides, and optionally esterification catalysts, such as described in U.S. Pat. No. 5,556,905, which is incorporated herein by reference, and in the case of ketones, by oxidation of their respective secondary alcohol precursors.

Benzoic acid derivatives from which solvents having the requisite solubility may be selected include: 1,4-cyclohexane dimethanol dibenzoate, diethylene glycol dibenzoate, dipropylene glycol dibenzoate, polypropylene glycol dibenzoate, propylene glycol dibenzoate, diethylene glycol benzoate and dipropylene glycol benzoate blend, polyethylene glycol (200) dibenzoate, iso-decyl benzoate, neo-pentyl glycol dibenzoate, glyceryl tribenzoate, pentaerylthritol tetrabenzoate, cumylphenyl benzoate, trimethyl pentanediol dibenzoate.

Phthalic acid derivatives from which solvents having the requisite solubility may be selected include: Alkyl benzyl phthalate, bis-cumyl-phenyl isophthalate, dibutoxyethyl phthalate, dimethyl phthalate, diethyl phthalate, dibutyl phthalate, diisobutyl phthalate, diisoheptyl phthalate, butyl octylphthalate, diisonoyl phthalate, nonyl undecylphthalate, dioctyl phthalate, di-isooctylphthalate, dicapryl phthalate, mixed alcohol phthalate, di-(2-ethylhexyl) phthalate, linear heptyl nonylphthalate8, linear heptyl nonyl undecyl phthalate, linear nonyl phthalate, linear nonyl undecylphthalate, linear dinonyl didecylphthalate, diisodecyl phthalate, diundecyl phthalate, ditridecyl phthalate, undecyidodecyl phthalate, decyltridecyl phthalate, 1:1 blend of dioctyl and didecyl phthalates, butyl benzyl phthalate, and dicyclohexyl phthalate.

Preferred solvents include the lower alkyl and aralkyl esters of the aryl acids described above. Examples of acids are benzoic acid and the phthalic acids, such as phthalic acid, isophthalic acid, and terephathalic acid. Most preferred solvents are derivatives of benzoic acid and include, but are not limited to, methyl benzoate, ethyl benzoate, n-propyl benzoate, isopropyl benzoate, butyl benzoate, isobutyl benzoate, sec-butyl benzoate, tert-butyl benzoate, isoamyl benzoate and benzyl benzoate, with ethyl benzoate being most especially preferred. Preferred solvent mixtures are those in which ethyl benzoate is the primary solvent, and mixtures formed of ethyl benzoate and either triacetin, tributyl citrate, triethyl citrate or N-methyl-2-pyrrolidone. Preferred mixtures are those in which ethyl benzoate is present by weight in an amount of 50% or more, more preferably 60% or more and most preferably 80% or more of the total amount of solvent present. Especially preferred mixtures are those of 80/20 mixtures by weight of ethyl benzoate/triacetin and ethyl benzoate/N-methyl-2-pyrrolidone.

The solvents described above having a miscibility in water of less than 7 wt % may be mixed with one or more additional miscible solvents, referred to as component solvents. Component solvents compatible and miscible with the primary solvent may have a higher miscibility with water, and the resulting mixtures may still exhibit significant control of water uptake into the implant. Such mixtures will be referred to as component solvent mixtures. Useful component solvent mixtures may exhibit solubilities in water greater than the primary solvents themselves, typically between 0.1 wt % and up to and including 50 wt %, preferably up to and including 30 wt %, and most preferably up to and including 10 wt %, without detrimentally affecting the control of water uptake exhibited by the implants of the invention. Especially preferred are component solvent mixtures having a solubility in water of about 0.1 wt % to about 7 wt %.

Component solvents useful in component solvent mixtures are those solvents that are miscible with the primary solvent or solvent mixture, and include, but are not limited to, triacetin, diacetin, tributyrin, triethyl citrate, tributyl citrate, acetyl triethyl citrate, acetyl tributyl citrate, triethylglycerides, triethyl phosphate, diethyl phthalate, diethyl tartrate, mineral oil, polybutene, silicone fluid, glylcerin, ethylene glycol, polyethylene glycol, octanol, ethyl lactate, propylene glycol, propylene carbonate, ethylene carbonate, butyrolactone, ethylene oxide, propylene oxide, N-methyl-2-pyrrolidone, 2-pyrrolidone, glycerol formal, methyl acetate, ethyl acetate, methyl ethyl ketone, dimethylformamide, dimethyl sulfoxide, tetrahydrofuran, caprolactam, decylmethylsulfoxide, oleic acid, 1-dodecylazacycloheptan-2-one, and mixtures thereof.

The solvent or solvent mixture is capable of dissolving or dispersing the polymer to form a viscous gel that can maintain particles of the beneficial agent dissolved or dispersed and isolated from the environment of use prior to release. Water uptake is regulated by the use of a solvent or component solvent mixture that solublizes or plasticizes the polymer but controls the uptake of water into the implant. In some cases, the solvent or component solvent mixture substantially restricts water uptake.

If the polymer composition is to be administered as an injectable gel, the level of polymer dissolution will affect the balance between the resulting gel viscosity and the potential for a premature burst. A premature burst is an initial dose of the bioactive agent released from the mixture during or shortly after injection. Highly viscous gels enable the beneficial agent to be delivered without exhibiting a significant premature burst effect but may make it difficult to dispense the gel through a needle. In those instances, an emulsifying agent or a component solvent may optionally be added to the composition to reduce the viscosity. Also, since the viscosity may generally be lowered as the temperature of the composition increases, it may be advantageous in certain applications to reduce the viscosity of the gel by heating to provide a more readily injectable composition.

When an emulsifying agent is mixed with the viscous gel formed from the polymer and the solvent using conventional static or mechanical mixing devices, such as an orifice mixer, the emulsifying agent forms a separate phase composed of dispersed droplets of microscopic size that typically have an average diameter of less than about 100 microns. The continuous phase is formed of the polymer and the solvent. The particles of the beneficial agent may be dissolved or dispersed in either the continuous phase or the droplet phase. In the resulting thixotropic composition, the droplets of emulsifying agent elongate in the direction of shear and substantially decrease the viscosity of the viscous gel formed from the polymer and the solvent. For instance, with a viscous gel having a viscosity of from about 5,000 to about 50,000 poise measured at 1.0 sec⁻¹ at 25° C., one can obtain a reduction in viscosity to less than 100 poise when emulsified with a 10% ethanol/water solution at 25° C. as determined by HAAKE Rheometer, available from THERMO HAAKE, Parmus, N.J.

When used, the emulsifying agent typically is present in an amount ranging from about 5 to about 80 wt %, preferably from about 20 to about 60 wt % and often 30 to 50 wt % based on the amount of the injectable depot gel composition, that is the combined amounts of polymer, solvent, emulsifying agent and beneficial agent. Emulsifying agents include, for example, solvents that are not fully miscible with the polymer solvent or solvent mixture. Examples of emulsifying agents include water, alcohols, polyols, esters, carboxylic acids, ketones, aldehydes and mixtures thereof. Preferred emulsifying agents are alcohols, propylene glycol, ethylene glycol, glycerol, water, and solutions and mixtures thereof. Especially preferred are water, ethanol, and isopropyl alcohol and solutions and mixtures thereof. The type of emulsifying agent affects the size of the dispersed droplets. For instance, ethanol will provide droplets that have average diameters that can be on the order of ten times larger than the droplets obtained with an isotonic saline solution containing 0.9% by weight of sodium chloride at 21° C.

It is to be understood that viscosity modifiers, in the form of emulsifying agents or component solvents, do not constitute mere diluents that reduce viscosity by simply decreasing the concentration of the components of the composition. The use of conventional diluents can reduce viscosity, but can also cause a premature burst effect when the diluted composition is injected. In contrast, an injectable depot composition can be formulated to avoid this premature burst effect by selecting the solvent and viscosity modifier such that once injected into place, the viscosity modifier has little impact on the release properties of the original system.

The solvent and any emulsifying agent used in the polymer mixture are preferably pharmaceutically acceptable carriers. The pharmaceutical compositions for the administration of the active compounds may conveniently be presented in dosage unit form and may be prepared by any of the methods well known in the art of pharmacy. All methods include the step of bringing the active compound into association with the carrier that constitutes one or more accessory ingredients. Preferably, the pharmaceutical compositions are prepared by uniformly and intimately bringing the active compound into association with a liquid carrier. In the pharmaceutical composition the active compound is included in an amount sufficient to produce the desired effect upon the process or condition of diseases.

Aqueous suspensions contain the active materials in admixture with excipients suitable for the manufacture of aqueous suspensions. Such excipients include suspending agents, for example sodium carboxymethylcellulose, methylcellulose, hydroxypropylmethylcellulose, sodium alginate, polyvinylpyrrolidone, gum tragacanth and gum acacia. Excipients may include dispersing or wetting agents, which may be a naturally-occurring phosphatide, for example lecithin; or condensation products of an alkylene oxide with fatty acids, for example polyoxyethylene stearate; or condensation products of ethylene oxide with long chain aliphatic alcohols, for example heptadecaethyleneoxycetanol; or condensation products of ethylene oxide with partial esters derived from fatty acids and a hexitol such as polyoxyethylene sorbitol monooleate; or condensation products of ethylene oxide with partial esters derived from fatty acids and hexitol anhydrides, for example polyethylene sorbitan monooleate. The aqueous suspensions may also contain one or more preservatives, for example ethyl- or n-propyl-p-hydroxybenzoate.

Oily suspensions may be formulated by suspending the active ingredient in a vegetable oil, for example arachis oil, olive oil, sesame oil or coconut oil; or in a mineral oil such as liquid paraffin. The oily suspensions may contain a thickening agent, for example beeswax, hard paraffin or cetyl alcohol. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients may also be present.

Pharmaceutical compositions may also be in the form of oil-in-water emulsions. The oily phase may be a vegetable oil, for example olive oil or arachis oil; or a mineral oil, for example liquid paraffin; or mixtures of these. Suitable emulsifying agents may be naturally occurring gums, for example gum acacia or gum tragacanth; naturally occurring phosphatides, for example soy bean and lecithin; esters or partial esters derived from fatty acids and hexitol anhydrides, for example sorbitan monooleate; or condensation products of the said partial esters with ethylene oxide, for example polyoxyethylene sorbitan monooleate.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in ethyl benzoate. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The beneficial agent can be any physiologically or pharmacologically active substance or substances optionally in combination with pharmaceutically acceptable carriers and additional ingredients such as antioxidants, stabilizing agents, permeation enhancers, etc. that do not substantially adversely affect the advantageous results that can be attained. The beneficial agent may be any of the agents which are known to be delivered to the body of a human or an animal and that are preferentially soluble in water rather than in the polymer-dissolving solvent. These agents include drug agents, medicaments, vitamins, nutrients, or the like. Included among the types of agents which meet this description are lower molecular weight compounds, proteins, peptides, genetic material, nutrients, vitamins, food supplements, sex sterilants, fertility inhibitors and fertility promoters.

Drug agents which may be delivered by the present invention include drugs which act on the peripheral nerves, adrenergic receptors, cholinergic receptors, the skeletal muscles, the cardiovascular system, smooth muscles, the blood circulatory system, synoptic sites, neuroeffector junctional sites, endocrine and hormone systems, the immunological system, the reproductive system, the skeletal system, autacoid systems, the alimentary and excretory systems, the histamine system and the central nervous system. Suitable agents may be selected from, for example, proteins, enzymes, hormones, polynucleotides, nucleoproteins, polysaccharides, glycoproteins, lipoproteins, polypeptides, steroids, analgesics, local anesthetics, antibiotic agents, anti-inflammatory corticosteroids, ocular drugs and synthetic analogs of these species.

Examples of drugs which may be delivered by the composition of the present invention include, but are not limited to, prochlorperzine edisylate, ferrous sulfate, aminocaproic acid, mecamylamine hydrochloride, procainamide hydrochloride, amphetamine sulfate, methamphetamine hydrochloride, benzamphetamine hydrochloride, isoproterenol sulfate, phenmetrazine hydrochloride, bethanechol chloride, methacholine chloride, pilocarpine hydrochloride, atropine sulfate, scopolamine bromide, isopropamide iodide, tridihexethyl chloride, phenformin hydrochloride, methylphenidate hydrochloride, theophylline cholinate, cephalexin hydrochloride, diphenidol, meclizine hydrochloride, prochlorperazine maleate, phenoxybenzamine, thiethylperzine maleate, anisindone, diphenadione erythrityl tetranitrate, digoxin, isoflurophate, acetazolamide, methazolamide, bendroflumethiazide, chloropromaide, tolazamide, chlormadinone acetate, phenaglycodol, allopurinol, aluminum aspirin, methotrexate, acetyl sulfisoxazole, erythromycin, hydrocortisone, hydrocorticosterone acetate, cortisone acetate, dexamethasone and its derivatives such as betamethasone, triamcinolone, methyltestosterone, 17-S-estradiol, ethinyl estradiol, ethinyl estradiol 3-methyl ether, prednisolone, 17-α-hydroxyprogesterone acetate, 19-norprogesterone, norgestrel, norethindrone, norethisterone, norethiederone, progesterone, norgesterone, norethynodrel, aspirin, indomethacin, naproxen, fenoprofen, sulindac, indoprofen, nitroglycerin, isosorbide dinitrate, propranolol, timolol, atenolol, alprenolol, cimetidine, clonidine, imipramine, levodopa, chlorpromazine, methyidopa, dihydroxyphenylalanine, theophylline, calcium gluconate, ketoprofen, ibuprofen, cephalexin, erythromycin, haloperidol, zomepirac, ferrous lactate, vincamine, diazepam, phenoxybenzamine, diltiazem, milrinone, mandol, quanbenz, hydrochlorothiazide, ranitidine, flurbiprofen, fenufen, fluprofen, tolmetin, alclofenac, mefenamic, flufenamic, difuinal, nimodipine, nitrendipine, nisoldipine, nicardipine, felodipine, lidoflazine, tiapamil, gallopamil, amlodipine, mioflazine, lisinolpril, enalapril, enalaprilat captopril, ramipril, famotidine, nizatidine, sucralfate, etintidine, tetratolol, minoxidil, chlordiazepoxide, diazepam, amitriptyline, and imipramine. Further examples are proteins and peptides which include, but are not limited to, bone morphogenic proteins, insulin, colchicine, glucagon, thyroid stimulating hormone, parathyroid and pituitary hormones, calcitonin, renin, prolactin, corticotrophin, thyrotropic hormone, follicle stimulating hormone, chorionic gonadotropin, gonadotropin releasing hormone, bovine somatotropin, porcine somatotropin, oxytocin, vasopressin, GRF, somatostatin, lypressin, pancreozymin, luteinizing hormone, LHRH, LHRH agonists and antagonists, leuprolide, interferons such as interferon alpha-2a, interferon alpha-2b, and consensus interferon, interleukins, growth hormones such as human growth hormone and its derivatives such as methione-human growth hormone and des-phenylalanine human growth hormone, bovine growth hormone and porcine growth hormone, fertility inhibitors such as the prostaglandins, fertility promoters, growth factors such as insulin-like growth factor, coagulation factors, human pancreas hormone releasing factor, analogs and derivatives of these compounds, and pharmaceutically acceptable salts of these compounds, or their analogs or derivatives.

The present invention also finds application with chemotherapeutic agents for the local application of such agents to avoid or minimize systemic side effects. Gels of the present invention containing chemotherapeutic agents may be injected directly into the tumor tissue for sustained delivery of the chemotherapeutic agent over time. In some cases, particularly after resection of the tumor, the gel may be implanted directly into the resulting cavity or may be applied to the remaining tissue as a coating. In cases in which the gel is implanted after surgery, it is possible to utilize gels having higher viscosities since they do not have to pass through a small diameter needle. Representative chemotherapeutic agents that may be delivered in accordance with the practice of the present invention include, for example, carboplatin, cisplatin, paclitaxel, BCNU, vincristine, camptothecin, etopside, cytokines, ribozymes, interferons, oligonucleotides and oligonucleotide sequences that inhibit translation or transcription of tumor genes, functional derivatives of the foregoing, and generally known chemotherapeutic agents such as those described in U.S. Pat. No. 5,651,986. The present application has particular utility in the sustained delivery of water soluble chemotherapeutic agents, such as for example cisplatin and carboplatin and the water soluble derivatives of paclitaxel.

To the extent not mentioned above, the beneficial agents described in aforementioned U.S. Pat. No. 5,242,910 can also be used. One particular advantage of the present invention is that materials, such as proteins, as exemplified by the enzyme lysozyme, and cDNA, and DNA incorporated into vectors both viral and nonviral, which are difficult to microencapsulate or process into microspheres can be incorporated into the compositions of the present invention without the level of degradation caused by exposure to high temperatures and denaturing solvents often present in other processing techniques.

The beneficial agent can be incorporated into the viscous gel formed from the polymer and the solvent in the form of particles typically having an average particle size of from about 0.1 to about 100 microns, or from about 1 to about 25 microns, or further from 2 to 10 microns. For instance, particles having an average particle size of about 5 microns have been produced by spray drying or freeze drying an aqueous mixture containing 50% sucrose and 50% chicken lysozyme (on a dry weight basis) and mixtures of 10-20% hGH and 15-30 mM zinc acetate. Conventional lyophilization processes can also be utilized to form particles of beneficial agents of varying sizes using appropriate freezing and drying cycles.

To form a suspension or dispersion of particles of the beneficial agent in the viscous gel formed from the polymer and the solvent, any conventional low shear device can be used such as a ROSS double planetary mixer (ROSS, Hauppauge, N.Y.) at ambient conditions. In this manner, efficient distribution of the beneficial agent can be achieved substantially without degrading the beneficial agent. The amount of beneficial agent in the mixture is preferably equal to a unit dosage. A unit dosage is the amount of beneficial agent necessary to produce the desired beneficial effect in the organism to which it is administered. The absolute amount of a unit dosage can depend on many factors including, for example, the type of agent, the efficacy of the agent, the health of the organism, and the size of the organism.

Mixtures as described may be administered in a variety of ways. Preferred methods of administration involve injection. Injection may be subcutaneous, parenteral, or other types of injection known to those skilled in the art. A consideration for administration by injection is the viscosity of the mixture. It is preferred that the viscosity is such that the mixture can be made to flow easily through an 18-20 gauge needle.

For an implant administered by injection, the fluid mixture transforms into a depot upon contact with the native fluid in the body. This depot is characterized by its phase separation from the physiological fluid and its decreased fluidity relative to the original mixture. The depot may be a semi-fluid gel, it may be a solid, or it may have an intermediate rigidity. It is this depot that serves as the polymeric implant for controlled release of the bioactive agent.

Since the implant systems of the present invention preferably are formed as viscous gels, the means of administration of the implants is not limited to injection, although that mode of delivery may often be preferred. Where the implant will be administered as a leave-behind product, it may be formed to fit into a body cavity existing after completion of surgery or it may be applied as a flowable gel by brushing or palleting the gel onto residual tissue or bone. Such applications may permit loading of beneficial agent in the gel at concentrations above those typically present in injectable compositions. It is also possible to form the depot outside the body and then to implant the depot surgically. In this case, the mixture can be formed, and then the solvent removed, for example by evaporation. Alternatively, the polymers can be extruded and layered.

Multi-layer, or composite, depots can also be formed. Composite depots can provide complex release profiles, including multiple stages of controlled burst release and controlled gradual release. Composite depots can be formed by supplying the injection needle with a mixture having a composition which changes during the injection process. For example, the initial composition may contain only amorphous polymer, the final composition may contain only crystallizable polymer, while the intermediate composition contains a polymer blend.

The composite formulation may be administered by injection of a composition that is itself layered. FIG. 11 illustrates a syringe 50 that is useful for preparing such layered injections. This syringe has two reservoirs 52 and 56 equipped with plungers 54 and 58 connected by a common handle 60. The reservoirs can be supplied with two different mixtures, for example a mixture containing amorphous polymer in one reservoir and a mixture containing crystallizable polymer in the other. A force on the handle in the direction of arrow 62 forces both mixtures into the dispensing area 64, which contains two chambers. The mixture in reservoir 52 is displaced into inner chamber 66, and the mixture in reservoir 56 is displaced into outer chamber 68 through tube 70. Both chambers are connected to opening 72 through which the layered mixture is dispensed. The opening 72 may be connected to a needle having a proper length and gauge to permit the layered mixture to be injected into an organism. Alternatively, the syringe 50 may be used to form a composite depot ex vivo, and the depot may be surgically inserted into an organism.

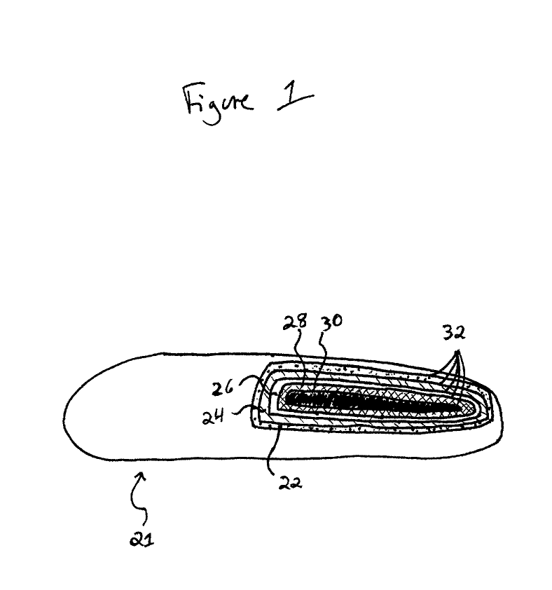

Composite depots can be formed by combining layers of a depot formulation ex vivo. For example, a process of depositing mixtures separately into a receptor buffer can be used to make a laminate, a layered disk, a series of concentric spheres, or any formed object. The formed depot may then be inserted surgically into the organism. An example of a composite depot is pictured in FIG. 1. Depot 21 may contain gel layers 22, 24, 26, 28, and 30. These layers may be separated by a barrier layer 32. This barrier layer may be a semi-permeable membrane such that water and bioactive agent can pass through the membrane, but the gel layers are prevented from mixing. This membrane is preferably biodegradable and insoluble in the solvent in the mixture. Alternatively, the barrier layer may be a biodegradable crystallizable polymer. For example, the exterior of a gel layer can be coated with a solution of poly(L-lactide) in dichloromethane. Evaporation of the volatile solvent will provide a layer of poly(L-lactide) around the gel layer. The poly(L-lactide) layer is biodegradable, but will dissolve very slowly or not at all in the biocompatible solvent present in the gel.

The gel layers in the composite depot 21 can have a variety of compositions. They may be alternated such that every other layer contains crystallizable polymer, and the remaining layers contain only amorphous polymer. They may all contain crystallizable polymer, and the type and relative amount of crystallizable polymer may be different or may independently be the same for at least two layers. A composite depot may contain at least one layer in which the solvent is miscible with water, providing for more rapid water uptake for that particular layer. The release profile for each layer will be affected by its composition. Thus, a complex release profile for the entire depot 21 can be designed by selection and ordering of the gel layers. Complex release profiles may be particularly effective for vaccinations, for example Hepatitis-B vaccine.

The amount and type of semi-crystalline polymer in the depot affects the release profile of the bioactive agent. For depots containing a sufficient concentration of semi-crystalline polymer, the bioactive agent is released from the depot very slowly or not at all for a period of time. This period of time could be as short as a few minutes, or as long as several weeks, or possibly several months. This lag time is followed by a rapid release of the bioactive agent. Depots containing no amorphous polymer can release almost all of the bioactive agent in this rapid release. Depots containing some amorphous polymer will release a portion of the bioactive agent in the rapid release, followed by a period of slow, gradual release.

The rapid release of the bioactive agent may coincide with the onset of crystallization of the semi-crystalline polymer. Because the bioactive agent is excluded from the polymer matrix, this mechanism of release can be more efficient overall than other controlled release systems which stop releasing the bioactive agent when an equilibrium concentration is reached. In some embodiments, almost all of the bioactive agent is released from the depot. Depots that exhibit the rapid release profile are further characterized by a porous structure. This structure develops during the crystallization process and may be a result of solid-liquid de-mixing.

For depots containing a sufficient concentration of amorphous polymer, the bioactive agent is released from the depot in a slow, gradual manner. These depots are characterized by a non-porous, or dense, structure even though crystallization may occur as observed by DSC. Phase separation may occur by liquid-liquid de-mixing, and the bioactive agent is released by diffusion through the depot.

The overall action of a biodegradable polymeric implant according to the invention can be widely varied, yet easily controllable. If the implanting mixture contains only amorphous polymer(s), the release of the bioactive agent will be slow and gradual. The conditions for these amorphous implants can be tailored to provide for zero-order release. If the implanting solution contains only semi-crystalline polymer(s), bioactive agent will be released in a rapid release. The type and relative amount of crystallizable polymer may affect the timing of the rapid release, that is the amount of time lag before the agent is released. The type and relative amount of crystallizable polymer may affect the amplitude of the rapid release, that is the amount of agent released during the rapid release.

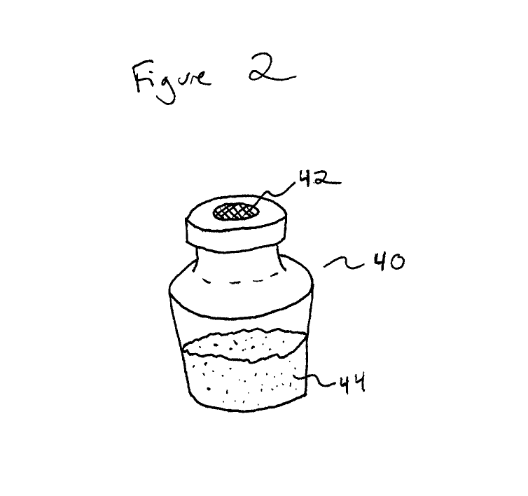

The mixture may be conveniently packaged in a sterile container, such as the vial 40 illustrated in FIG. 2. This container may be part of a kit which may optionally contain a sterile syringe and needle. The vial 40 may be sealed with a septum 42. This septum seals the mixture 44 and may be pierced by a needle and syringe to permit withdrawal of the mixture. The vial may contain all the ingredients necessary for the controlled release of the bioactive agent. It is preferred that end user of the mixture not be required to add further ingredients or to measure the dosage prior to administration.

### EXAMPLES

Poly(ε-caprolactone) (PCL) was obtained from ALDRICH, Milwaukee, Wis., with a reported molecular weight of 14,000 daltons. Poly(D,L-lactide) (PDLA) was obtained from BOEHRINGER INGELHEIM, Ridgefield, Conn., as RESOMER 202, with a reported molecular weight of 12,000 daltons. Ethyl benzoate was reagent grade purity from ALDRICH, Milwaukee, Wis. Chicken egg white lysozyme was used as the model bioactive agent released from the depot. Specifically, the lysozyme was Muramidase, or mucopeptide N-acetylmuramoyl-hydrolase, Enzyme Commission Number 3.2.1.17, obtained from SIGMA, Milwaukee, Wis. The activity of the lysozyme was approximately 50,000 units per milligram protein. The receptor solution was a phosphate buffered saline (PBS) of pH 7.4.

### Example 1

A polymer mixture was prepared by mixing PCL with ethyl benzoate in a 10 cm³ glass vial. In order to effect good solubilization, the mixture was sealed and heated at 50° C. for 24 to 48 hours. The mixture was periodically stirred to disperse lumps and remove trapped air bubbles. Once the polymer was dissolved completely, the solution was cooled to 37° C. and stored. Lysozyme particles were added to the polymer solution at a level of 10% by weight of the total formulation. Composition of the mixture is given in Table 1.

### Examples 2-4

Polymer blends were prepared as in Example 1, but using a mixture of PCL and PDLA as the polymeric component. Lysozyme particles were added to the polymer blend solutions. Compositions of the mixtures are given in Table 1.

### Example 5

A polymer mixture was prepared as in Example 1, but using PDLA as the polymeric component. Lysozyme particles were added to the polymer solution. Composition of the mixture is given in Table 1.

TABLE 1

Composition in weight percent

| Example | PCL | PDLA | Ethyl benzoate | Lysozyme |
|---|---|---|---|---|
| 1 | 45 | 0 | 45 | 10 |
| 2 | 31.5 | 13.5 | 45 | 10 |
| 3 | 22.5 | 22.5 | 45 | 10 |
| 4 | 13.5 | 31.5 | 45 | 10 |
| 5 | 0 | 45 | 45 | 10 |

Crystallization Behavior

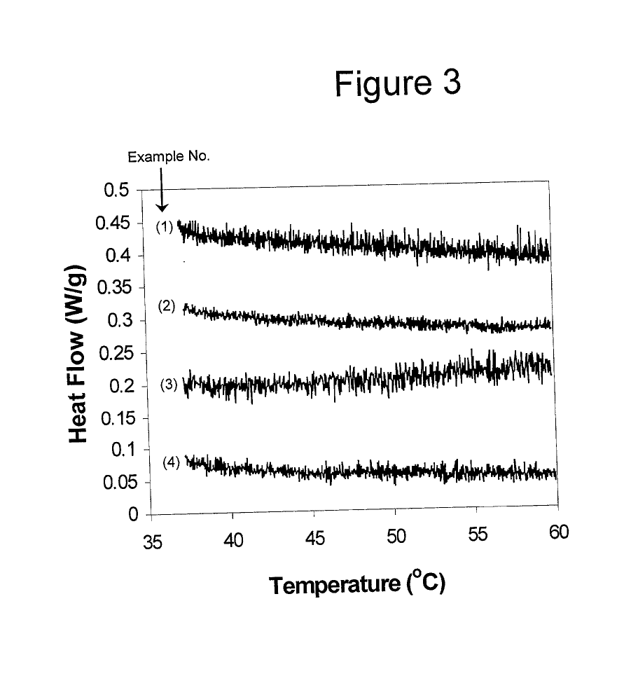

Solutions from Examples 1-4 were analyzed by differential scanning calorimetry (DSC) using a PERKIN ELMER DSC-7 instrument (PERKIN ELMER INSTRUMENTS, Boston, Mass.). The solutions were kept at 37° C. until they were placed in the calorimeter. Data were collected from 37° C. to 60° C. at a heating rate of 1° C./minute. The scans are shown in FIG. 3. None of the samples gives evidence of a Tₘ.

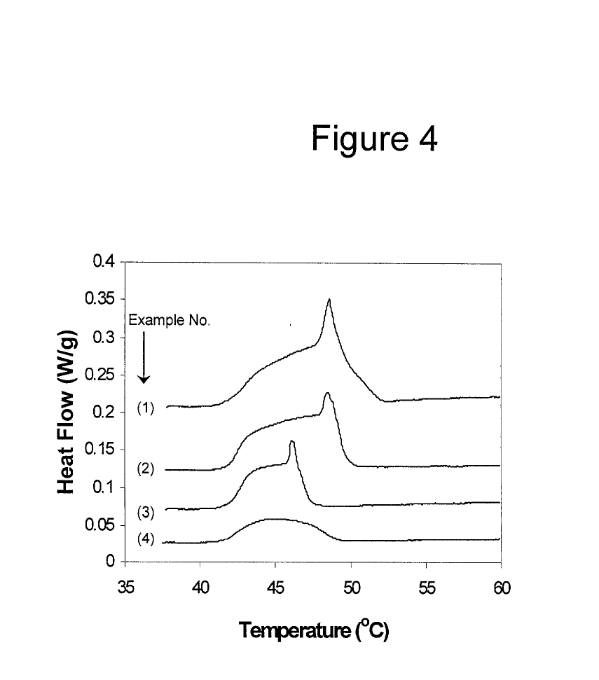

The solutions were separately injected via syringe into an agitated, temperature-controlled PBS bath at 37° C. to form depots. After 4 days, the depots were removed from the bath, and a small sample was taken from the surface. The samples were maintained at 37° C. until they were placed in the calorimeter. Data were collected as for the solutions, and the scans are shown in FIG. 4. All four samples show an endothermic transition indicative of a Tₘ.

Protein Release

Protein release experiments were conducted in a HANSON RESEARCH, SR8-PLUS dissolution test station (HANSON RESEARCH, Chatsworth, Calif.) configured as a USP Apparatus 1, with the modification of 100 mL flasks. Approximately 0.5 g of each formulation (Examples 1-5) was placed in a separate mesh basket and then quenched in 50 mL portions of PBS receptor solution. The samples were maintained at 37° C. and continuously rotated at 100 rpm. Deionized water was added to the receptor solution daily to account for evaporation. Aliquots of 200 μL were removed every 1-2 days to analyze for protein content. Polypropylene collection vials were used to minimize protein adsorption.

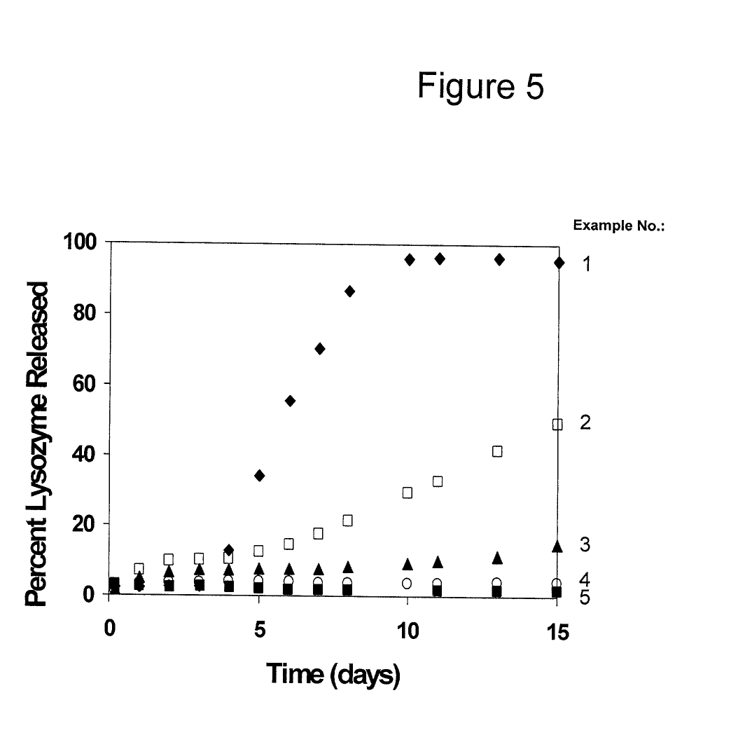

The aliquots were analyzed using high performance liquid chromatography (HPLC) utilizing a YMC brand reversed phase column (WATERS, Milford, Mass., Part No.: MA12S030546WT) with a UV detection at 210 nm in a WATERS 2690-D separation module. Lysozyme concentrations were determined using a calibration curve of standards ranging from 5 to 500 μg/mL. Two determinations were made for each release experiment. Data for protein release over time are given in FIG. 5.

Morphologies of Depots

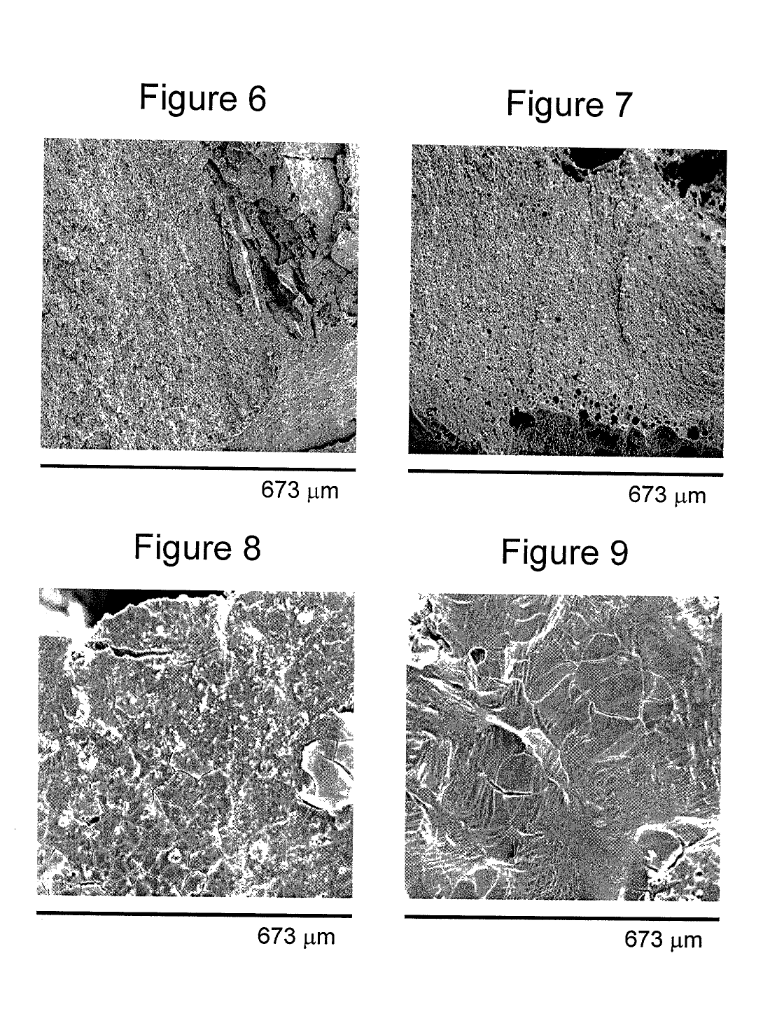

Morphologies of the fully solidified depots were examined using scanning electron microscopy (SEM). Samples were prepared by injecting approximately 0.5 g of polymer solution into an aqueous bath at 37° C. The solutions formed depots in the shape of disks of approximately 1.3 cm diameter and 0.3 cm thickness. The disks were removed from the bath after 15 days, fractured in liquid nitrogen, and dried under vacuum at room temperature for at least 24 hours. Samples for microscopy were sputter coated with an Au/Pd mixture using an EMSCOPE SC400 sputter coater. Micrographs were taken using a HITACHI S-530 SEM (HITACHI, LTD., Tokyo, Japan). The micrographs are shown in FIGS. 6-9 for Examples 1, 2, 3, and 4, respectively.

Bulk Water Absorption

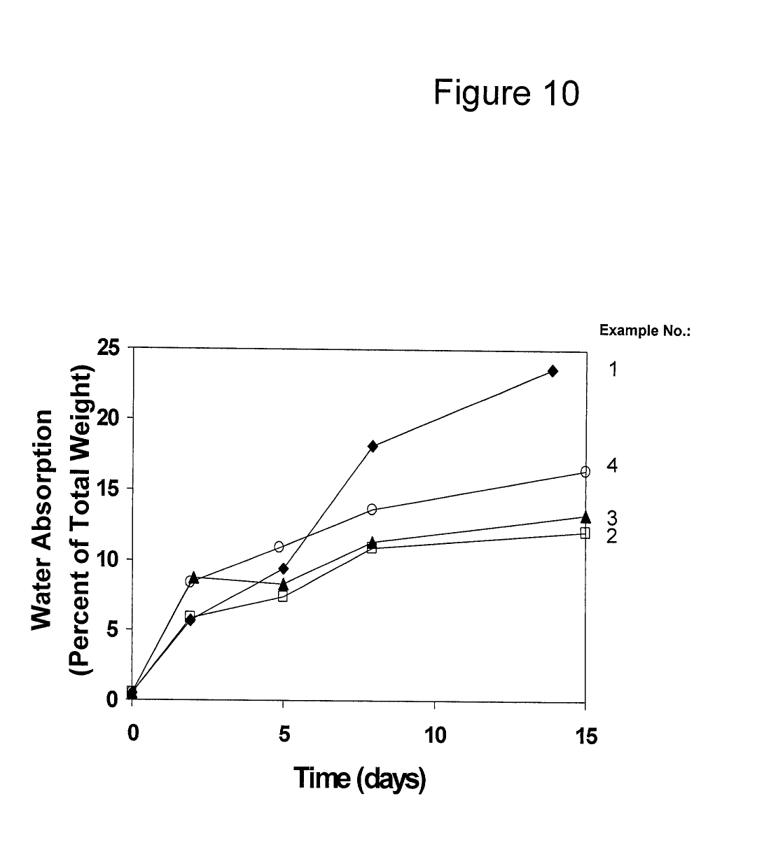

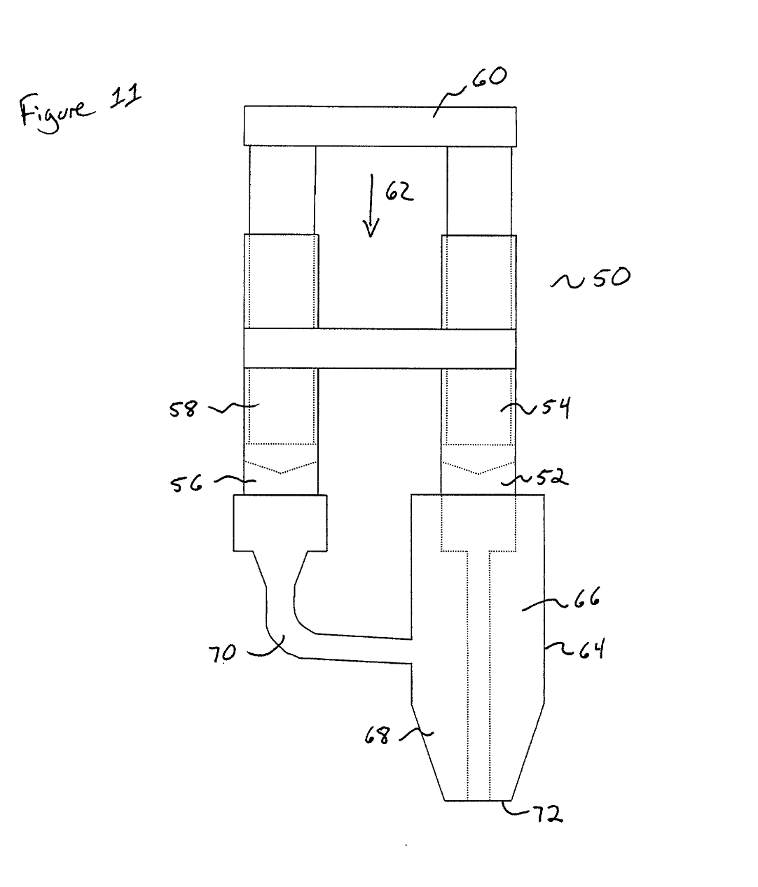

Bulk water absorption in the depot formulations was measured using Karl Fischer titrations on a METTLER TOLEDO DL31 titrator (METTLER TOLEDO, Columbus, Ohio). Measurements were repeated for each sample. Samples were prepared as in the protein release experiments. Data for water absorption by the depots are given in FIG. 10.

1. A composition for controlled release of a bioactive agent, comprising:
a biodegradable crystallizable polymer;
a biocompatible solvent; and
a bioactive agent.

2. The composition of claim 1, wherein the solvent has a miscibility with water less than 7 percent by weight.

3. The composition of claim 1, further comprising at least one biocompatible component solvent.

4. The composition of claim 1, further comprising an emulsifying agent.

5. The composition of claim 1, wherein the composition is sterile.

6. The composition of claim 1, wherein the biodegradable crystallizable polymer is a polyester.

7. The composition of claim 1, wherein the biodegradable crystallizable polymer is poly(ε-caprolactone).

8. The composition of claim 1, wherein the biocompatible solvent is ethyl benzoate.

9. The composition of claim 1, further comprising a biodegradable amorphous polymer.

10. The composition of claim 9, wherein the solvent has a miscibility with water less than 7 percent by weight.

11. The composition of claim 9, further comprising at least one biocompatible component solvent.

12. The composition of claim 9, further comprising an emulsifying agent.

13. The composition of claim 9, wherein the biocompatible solvent is ethyl benzoate.

14. The composition of claim 9, wherein the composition is sterile.

15. The composition of claim 9, wherein the biodegradable crystallizable polymer is a polyester.

16. The composition of claim 9, wherein the biodegradable crystallizable polymer is poly(ε-caprolactone).

17. The composition of claim 9, wherein the biodegradable amorphous polymer is a polyester.

18. The composition of claim 9, wherein the biodegradable amorphous polymer is poly(D,L-lactide).

19. The composition of claim 18, wherein the biodegradable crystallizable polymer is poly(ε-caprolactone) and the biocompatible solvent is ethyl benzoate.

20. The composition of claim 1, wherein the composition is multi-layered.

21. A composition for controlled release of a bioactive agent, comprising: a biodegradable crystallizable polymer and a biodegradable amorphous polymer.

22. The composition of claim 21, further comprising a biocompatible solvent.

23. The composition of claim 22, wherein the solvent has a miscibility with water less than 7 percent by weight.

24. The composition of claim 22, wherein the solvent is ethyl benzoate.

25. The composition of claim 22, further comprising at least one biocompatible component solvent.

26. The composition of claim 22, further comprising an emulsifying agent.

27. The composition of claim 21, further comprising a bioactive agent.

28. The composition of claim 21, wherein the composition is sterile.

29. The composition of claim 21, wherein the biodegradable crystallizable polymer is a polyester.

30. The composition of claim 21, wherein the biodegradable crystallizable polymer is poly(ε-caprolactone).

31. The composition of claim 21, wherein the biodegradable amorphous polymer is a polyester.

32. The composition of claim 21, wherein the biodegradable amorphous polymer is poly(D,L-lactide).

33. The composition of claim 24, wherein the biodegradable crystallizable polymer is poly(ε-caprolactone) and the biodegradable amorphous polymer is poly(D,L-lactide).

34. A method of administering a bioactive agent, comprising: inserting the composition of claim 1 into an organism.

35. The method of claim 34, wherein the inserting is by injecting.

36. A method of administering a bioactive agent, comprising: inserting the composition of claim 21 into an organism.

37. The method of claim 36, wherein the inserting is by injecting.

38. A method of making the composition of claim 1, comprising: combining ingredients;
wherein said ingredients comprise a biodegradable crystallizable polymer; a biocompatible solvent; and a bioactive agent.

39. The method of claim 38, wherein the ingredients further comprise a biodegradable amorphous polymer.

40. A method of making the composition of claim 21, comprising: combining ingredients;
wherein said ingredients comprise a biodegradable crystallizable polymer and a biodegradable amorphous polymer.

41. The method of claim 40, wherein the ingredients further comprise a bioactive agent.

42. A kit, comprising:
a container; and
a mixture, in said container, comprising a biodegradable crystallizable polymer, a bioactive agent, and a biocompatible solvent.

43. The kit of claim 42, wherein the mixture comprises a unit dosage of said bioactive agent.

44. The kit of claim 42, wherein the mixture is sterile.

45. The kit of claim 42, wherein the mixture further comprises a biodegradable amorphous polymer.

46. The kit of claim 42, further comprising a syringe.

47. The kit of claim 42, wherein the container comprises a septum.
